(19) United States
(12) Patent Application Publication
(10) Pub. No.: US 2026/0334563 A1
(43) Pub. Date: Oct. 29, 2026

(54) AEROSOL DELIVERY SYSTEMS AND METHODS

(71) Applicant: Nicoventures Trading Limited, London (GB)

(72) Inventor: Howard ROTHWELL, London (GB)

(21) Appl. No.: 19/122,224

(22) PCT Filed: Oct. 17, 2023
(86) PCT No.: PCT/GB2023/052691

§ 371 Date: Apr. 17, 2025

(30) Foreign Application Priority Data
Oct. 18, 2022 (GB) 2215363.9

(51) Int. Cl. A24F 40/485; A24F 40/10; A24F 40/42; A24F 40/50; A61M 15/06

(52) U.S. Cl. CPC A24F 40/485; A24F 40/42; A24F 40/50; A61M 15/06; A24F 40/10; A61M 2206/14

(57) ABSTRACT

An aerosol delivery subsystem comprising a fluid channel wall defining a fluid channel, wherein at least a portion of the fluid channel tapers along its length; and a flow modifying member in the tapering portion of the fluid channel, configured to impart a degree of rotation about the axis of extent of the fluid channel to aerosol flowing in the fluid channel in use.

### CROSS-REFERENCE TO RELATED APPLICATIONS

This present application is a National Phase entry of PCT Application No. PCT/GB2023/052691, filed Oct. 17, 2023, which claims priority from Great Britain Application No. 2215363.9, filed Oct. 18, 2022, each of which are fully incorporated herein by reference in their entireties.

### FIELD

The present disclosure relates to aerosol delivery systems such as, but not exclusively, nicotine delivery systems (e.g. e-cigarettes).

### BACKGROUND

Aerosol delivery systems such as electronic cigarettes (e-cigarettes) generally contain an aerosol generating material, such as a chamber of a source solid or liquid, which may contain an active substance and/or a flavour, from which an aerosol or vapour is generated for inhalation by a user, for example through heat vaporisation. Thus, an aerosol delivery system will typically comprise an aerosol generation area containing an aerosol generator, e.g. a heating element, arranged to vaporise or aerosolise a portion of precursor material to generate a vapour or aerosol in the aerosol generation area. As a user inhales on the device and electrical power is supplied to the vaporiser, air is drawn into the device through an inlet hole and along an inlet air channel connecting to the aerosol generation area, where the air mixes with vaporised precursor material to form a condensation aerosol. There is an outlet channel connecting the aerosol generation area to an outlet in the mouthpiece and the air drawn into the aerosol generation area as a user inhales on the mouthpiece continues along the outlet flow path to the mouthpiece outlet, carrying the aerosol with it, for inhalation by the user. Some electronic cigarettes may also include a flavour element in the air flow path through the device to impart additional flavours. Such devices may sometimes be referred to as hybrid devices, and the flavour element may, for example, include a portion of tobacco arranged in the air flow path between the aerosol generation area and the mouthpiece such that aerosol/condensation aerosol drawn through the device passes through the portion of tobacco before exiting the mouthpiece for user inhalation.

User experiences with electronic aerosol delivery systems are continually improving as such systems become more refined in respect of the nature of the vapour they provide for user inhalation, for example in terms of deep lung delivery, mouth feel and consistency in performance. Nonetheless, approaches for improving further still on these aspects remain of interest. In particular, it is of interest to develop approaches in which an aerosol delivery system comprises functionality enabling operating characteristics of the system to be adjusted, preferably automatically, in order to target certain operating characteristics which may be desirable to a user.

Various approaches are described herein which seek to help address or mitigate at least some of the issues discussed above.

### Terminology

### Delivery System

As used herein, the term “delivery system” is intended to encompass systems that deliver at least one substance to a user in use, and includes:
combustible aerosol provision systems, such as cigarettes, cigarillos, cigars, and tobacco for pipes or for roll-your-own or for make-your-own cigarettes (whether based on tobacco, tobacco derivatives, expanded tobacco, reconstituted tobacco, tobacco substitutes or other smokable material);
non-combustible aerosol provision systems that release compounds from an aerosol-generating material without combusting the aerosol-generating material, such as electronic cigarettes, tobacco heating products, and hybrid systems to generate aerosol using a combination of aerosol-generating materials; and
aerosol-free delivery systems that deliver the at least one substance to a user orally, nasally, transdermally or in another way without forming an aerosol, including but not limited to, lozenges, gums, patches, articles comprising inhalable powders, and oral products such as oral tobacco which includes snus or moist snuff, wherein the at least one substance may or may not comprise nicotine.

### Combustible Aerosol Provision System

According to the present disclosure, a “combustible” aerosol provision system is one where a constituent aerosol-generating material of the aerosol provision system (or component thereof) is combusted or burned during use in order to facilitate delivery of at least one substance to a user.

In some embodiments, the delivery system is a combustible aerosol provision system, such as a system selected from the group consisting of a cigarette, a cigarillo and a cigar.

In some embodiments, the disclosure relates to a component for use in a combustible aerosol provision system, such as a filter, a filter rod, a filter segment, a tobacco rod, a spill, an aerosol-modifying agent release component such as a capsule, a thread, or a bead, or a paper such as a plug wrap, a tipping paper or a cigarette paper.

### Non-Combustible Aerosol Provision System

According to the present disclosure, a “non-combustible” aerosol provision system is one where a constituent aerosol-generating material of the aerosol provision system (or component thereof) is not combusted or burned in order to facilitate delivery of at least one substance to a user.

In some embodiments, the delivery system is a non-combustible aerosol provision system, such as a powered non-combustible aerosol provision system.

In some embodiments, the non-combustible aerosol provision system is an electronic cigarette, also known as a vaping device or electronic nicotine delivery system (END), although it is noted that the presence of nicotine in the aerosol-generating material is not a requirement.

In some embodiments, the non-combustible aerosol provision system is an aerosol-generating material heating system, also known as a heat-not-burn system. An example of such a system is a tobacco heating system.

In some embodiments, the non-combustible aerosol provision system is a hybrid system to generate aerosol using a combination of aerosol-generating materials, one or a plurality of which may be heated. Each of the aerosol-generating materials may be, for example, in the form of a solid, liquid or gel and may or may not contain nicotine. In some embodiments, the hybrid system comprises a liquid or gel aerosol-generating material and a solid aerosol-generating material. The solid aerosol-generating material may comprise, for example, tobacco or a non-tobacco product.

Typically, the non-combustible aerosol provision system may comprise a non-combustible aerosol provision device and a consumable for use with the non-combustible aerosol provision device.

In some embodiments, the disclosure relates to consumables comprising aerosol-generating material and configured to be used with non-combustible aerosol provision devices. These consumables are sometimes referred to as articles throughout the disclosure.

In some embodiments, the non-combustible aerosol provision system, such as a non-combustible aerosol provision device thereof, may comprise a power source and a controller. The power source may, for example, be an electric power source or an exothermic power source. In some embodiments, the exothermic power source comprises a carbon substrate which may be energised so as to distribute power in the form of heat to an aerosol-generating material or to a heat transfer material in proximity to the exothermic power source.

In some embodiments, the non-combustible aerosol provision system may comprise an area for receiving the consumable, an aerosol generator, an aerosol generation area, a housing, a mouthpiece, a filter and/or an aerosol-modifying agent.

In some embodiments, the consumable for use with the non-combustible aerosol provision device may comprise aerosol-generating material, an aerosol-generating material storage area, an aerosol-generating material transfer component, an aerosol generator, an aerosol generation area, a housing, a wrapper, a filter, a mouthpiece, and/or an aerosol-modifying agent.

### Aerosol-Free Delivery System

In some embodiments, the delivery system is an aerosol-free delivery system that delivers at least one substance to a user orally, nasally, transdermally or in another way without forming an aerosol, including but not limited to, lozenges, gums, patches, articles comprising inhalable powders, and oral products such as oral tobacco which includes snus or moist snuff, wherein the at least one substance may or may not comprise nicotine.

In some embodiments, the substance to be delivered may be an aerosol-generating material or a material that is not intended to be aerosolised. As appropriate, either material may comprise one or more active constituents, one or more flavours, one or more aerosol-former materials, and/or one or more other functional materials.

### Active Substance

In some embodiments, the substance to be delivered comprises an active substance. The active substance as used herein may be a physiologically active material, which is a material intended to achieve or enhance a physiological response. The active substance may for example be selected from nutraceuticals, nootropics, psychoactives. The active substance may be naturally occurring or synthetically obtained. The active substance may comprise for example nicotine, caffeine, taurine, theine, vitamins such as B6 or B12 or C, melatonin, cannabinoids, or constituents, derivatives, or combinations thereof. The active substance may comprise one or more constituents, derivatives or extracts of tobacco, cannabis or another botanical.

In some embodiments, the active substance comprises nicotine. In some embodiments, the active substance comprises caffeine, melatonin or vitamin B12.

As noted herein, the active substance may comprise one or more constituents, derivatives or extracts of cannabis, such as one or more cannabinoids or terpenes.

As noted herein, the active substance may comprise or be derived from one or more botanicals or constituents, derivatives or extracts thereof. As used herein, the term “botanical” includes any material derived from plants including, but not limited to, extracts, leaves, bark, fibres, stems, roots, seeds, flowers, fruits, pollen, husk, shells or the like. Alternatively, the material may comprise an active compound naturally existing in a botanical, obtained synthetically. The material may be in the form of liquid, gas, solid, powder, dust, crushed particles, granules, pellets, shreds, strips, sheets, or the like.

Example botanicals are tobacco, eucalyptus, star anise, hemp, cocoa, cannabis, fennel, lemongrass, peppermint, spearmint, rooibos, chamomile, flax, ginger, ginkgo biloba, hazel, hibiscus, laurel, licorice (liquorice), matcha, mate, orange skin, papaya, rose, sage, tea such as green tea or black tea, thyme, clove, cinnamon, coffee, aniseed (anise), basil, bay leaves, cardamom, coriander, cumin, nutmeg, oregano, paprika, rosemary, saffron, lavender, lemon peel, mint, juniper, elderflower, vanilla, wintergreen, beefsteak plant, curcuma, turmeric, sandalwood, cilantro, bergamot, orange blossom, myrtle, cassis, valerian, pimento, mace, damien, marjoram, olive, lemon balm, lemon basil, chive, carvi, verbena, tarragon, geranium, mulberry, ginseng, theanine, theacrine, maca, ashwagandha, damiana, guarana, chlorophyll, baobab or any combination thereof. The mint may be chosen from the following mint varieties: Mentha Arventis, Mentha c.v., Mentha niliaca, Mentha piperita, Mentha piperita citrata c.v., Mentha piperita c.v, Mentha spicata crispa, Mentha cardifolia, Memtha longifolia, Mentha suaveolens variegata, Mentha pulegium, Mentha spicata c.v. and Mentha suaveolens.

In some embodiments, the active substance comprises or is derived from one or more botanicals or constituents, derivatives or extracts thereof and the botanical is tobacco. In some embodiments, the active substance comprises or derived from one or more botanicals or constituents, derivatives or extracts thereof and the botanical is selected from eucalyptus, star anise, cocoa and hemp.

In some embodiments, the active substance comprises or derived from one or more botanicals or constituents, derivatives or extracts thereof and the botanical is selected from rooibos and fennel.

### Flavours

In some embodiments, the substance to be delivered comprises a flavour. As used herein, the terms “flavour” and “flavourant” refer to materials which, where local regulations permit, may be used to create a desired taste, aroma or other somatosensorial sensation in a product for adult consumers. They may include naturally occurring flavour materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavour enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, liquid such as an oil, solid such as a powder, or gas.

In some embodiments, the flavour comprises menthol, spearmint and/or peppermint. In some embodiments, the flavour comprises flavour components of cucumber, blueberry, citrus fruits and/or redberry. In some embodiments, the flavour comprises eugenol. In some embodiments, the flavour comprises flavour components extracted from tobacco. In some embodiments, the flavour comprises flavour components extracted from cannabis.

In some embodiments, the flavour may comprise a sensate, which is intended to achieve a somatosensorial sensation which are usually chemically induced and perceived by the stimulation of the fifth cranial nerve (trigeminal nerve), in addition to or in place of aroma or taste nerves, and these may include agents providing heating, cooling, tingling, numbing effect. A suitable heat effect agent may be, but is not limited to, vanillyl ethyl ether and a suitable cooling agent may be, but not limited to eucolyptol, WS-3.

### Aerosol-Generating Material

Aerosol-generating material is a material that is capable of generating aerosol, for example when heated, irradiated or energized in any other way. Aerosol-generating material may, for example, be in the form of a solid, liquid or gel which may or may not contain an active substance and/or flavourants. In some embodiments, the aerosol-generating material may comprise an “amorphous solid”, which may alternatively be referred to as a “monolithic solid” (i.e. non-fibrous). In some embodiments, the amorphous solid may be a dried gel. The amorphous solid is a solid material that may retain some fluid, such as liquid, within it. In some embodiments, the aerosol-generating material may for example comprise from about 50 wt %, 60 wt % or 70 wt % of amorphous solid, to about 90 wt %, 95 wt % or 100 wt % of amorphous solid.

The aerosol-generating material may comprise one or more active substances and/or flavours, one or more aerosol-former materials, and optionally one or more other functional material.

### Aerosol-Former Material

The aerosol-former material may comprise one or more constituents capable of forming an aerosol. In some embodiments, the aerosol-former material may comprise one or more of glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate.

### Functional Material

The one or more other functional materials may comprise one or more of pH regulators, colouring agents, preservatives, binders, fillers, stabilizers, and/or antioxidants.

### Substrate

The material may be present on or in a support, to form a substrate. The support may, for example, be or comprise paper, card, paperboard, cardboard, reconstituted material, a plastics material, a ceramic material, a composite material, glass, a metal, or a metal alloy. In some embodiments, the support comprises a susceptor. In some embodiments, the susceptor is embedded within the material. In some alternative embodiments, the susceptor is on one or either side of the material.

### Consumable

A consumable is an article comprising or consisting of aerosol-generating material, part or all of which is intended to be consumed during use by a user. A consumable may comprise one or more other components, such as an aerosol-generating material storage area, an aerosol-generating material transfer component, an aerosol generation area, a housing, a wrapper, a mouthpiece, a filter and/or an aerosol-modifying agent. A consumable may also comprise an aerosol generator, such as a heater, that emits heat to cause the aerosol-generating material to generate aerosol in use. The heater may, for example, comprise combustible material, a material heatable by electrical conduction, or a susceptor.

### Susceptor

A susceptor is a material that is heatable by penetration with a varying magnetic field, such as an alternating magnetic field. The susceptor may be an electrically-conductive material, so that penetration thereof with a varying magnetic field causes induction heating of the heating material. The heating material may be magnetic material, so that penetration thereof with a varying magnetic field causes magnetic hysteresis heating of the heating material. The susceptor may be both electrically-conductive and magnetic, so that the susceptor is heatable by both heating mechanisms. The device that is configured to generate the varying magnetic field is referred to as a magnetic field generator, herein.

### Aerosol-Modifying Agent

An aerosol-modifying agent is a substance, typically located downstream of the aerosol generation area, that is configured to modify the aerosol generated, for example by changing the taste, flavour, acidity or another characteristic of the aerosol. The aerosol-modifying agent may be provided in an aerosol-modifying agent release component, that is operable to selectively release the aerosol-modifying agent. The aerosol-modifying agent may, for example, be an additive or a sorbent. The aerosol-modifying agent may, for example, comprise one or more of a flavourant, a colourant, water, and a carbon adsorbent. The aerosol-modifying agent may, for example, be a solid, a liquid, or a gel. The aerosol-modifying agent may be in powder, thread or granule form. The aerosol-modifying agent may be free from filtration material.

### Aerosol Generator

An aerosol generator is an apparatus configured to cause aerosol to be generated from the aerosol-generating material. In some embodiments, the aerosol generator is a heater configured to subject the aerosol-generating material to heat energy, so as to release one or more volatiles from the aerosol-generating material to form an aerosol. In some embodiments, the aerosol generator is configured to cause an aerosol to be generated from the aerosol-generating material without heating. For example, the aerosol generator may be configured to subject the aerosol-generating material to one or more of vibration, increased pressure, or electrostatic energy.

The present disclosure relates to aerosol delivery systems (which may also be referred to as vapour delivery systems) such as nebulisers or e-cigarettes. Throughout the following description the term “e-cigarette” or “electronic cigarette” may sometimes be used, but it will be appreciated this term may be used interchangeably with aerosol delivery system/device and electronic aerosol delivery system/device. Furthermore, and as is common in the technical field, the terms “aerosol” and “vapour”, and related terms such as “vaporise”, “volatilise” and “aerosolise”, may generally be used interchangeably.

Aerosol delivery systems (e-cigarettes) often, though not always, comprise a modular assembly comprising a reusable device part and a replaceable (disposable/consumable) cartridge part. Often, the replaceable cartridge part will comprise the aerosol generating material and the vaporiser (which may collectively be called a ‘cartomizer’) and the reusable device part will comprise the power supply (e.g. rechargeable power source) and control circuitry. It will be appreciated these different parts may comprise further elements depending on functionality. For example, the reusable device part will often comprise a user interface for receiving user input and displaying operating status characteristics, and the replaceable cartridge device part in some cases comprises a temperature sensor for helping to control temperature. Cartridges are electrically and mechanically coupled to the control unit for use, for example using a screw thread, bayonet, or magnetic coupling with appropriately arranged electrical contacts. When the aerosol generating material in a cartridge is exhausted, or the user wishes to switch to a different cartridge having a different aerosol generating material, the cartridge may be removed from the reusable part and a replacement cartridge attached in its place. Systems and devices conforming to this type of two-part modular configuration may generally be referred to as two-part systems/devices.

It is common for electronic cigarettes to have a generally elongate shape. For the sake of providing a concrete example, certain embodiments of the disclosure will be taken to comprise this kind of generally elongate two-part system employing disposable cartridges. However, it will be appreciated that the underlying principles described herein may equally be adopted for different configurations, for example single-part systems or modular systems comprising more than two parts, refillable devices and single-use disposables, as well as other overall shapes, for example based on so-called box-mod high performance devices that typically have a boxier shape. More generally, it will be appreciated certain embodiments of the disclosure are based on aerosol delivery systems which are operationally configured to provide functionality in accordance with the principles described herein and the constructional aspects of systems configured to provide the functionality in accordance with certain embodiments of the disclosure is not of primary significance.

### BRIEF SUMMARY OF THE INVENTION

The present invention provides an aerosol delivery subsystem for an aerosol delivery system, comprising: a fluid channel wall defining a fluid channel, wherein at least a portion of the fluid channel tapers along its length; and a flow modifying member in the tapering portion of the fluid channel, configured to impart a degree of rotation about an axis of extent of the fluid channel to aerosol flowing in the fluid channel in use.

The present invention further provides a flow modifying device for an aerosol delivery system, the device comprising: a fluid channel wall defining a fluid channel, wherein at least a portion of the fluid channel tapers along its length; and a flow modifying member in the tapering portion of the fluid channel, configured to impart a degree of rotation about an axis of extent of the fluid channel to aerosol flowing in the fluid channel in use.

The present invention further provides an aerosol delivery subsystem, comprising: a fluid channel means defining a fluid channel, wherein at least a portion of the fluid channel tapers along its length; and a flow modifying means in the tapering portion of the fluid channel, configured to impart a degree of rotation about the axis of extent of the fluid channel to aerosol flowing in the fluid channel in use.

The present invention further provides a method of manufacturing an aerosol delivery subsystem, comprising providing: a fluid channel wall defining a fluid channel, wherein at least a portion of the fluid channel tapers along its length; and a flow modifying member in the tapering portion of the fluid channel, configured to impart a degree of rotation about the axis of extent of the fluid channel to aerosol flowing in the fluid channel in use.

The present invention further provides a method of modifying fluid flow along a flow channel defined by a fluid channel wall, wherein at least a portion of the fluid channel tapers along its length, the method comprising: imparting a degree of rotation about the axis of extent of the fluid channel to aerosol flowing in the tapering portion of the fluid channel.

The present invention also provides preferred embodiments as claimed in the dependent claims.

The claimed invention generally provides a flow modifying device or sub-assembly suitable for use in an aerosol delivery system, or configured for use in an aerosol delivery system. The flow modifying device or sub-assembly may generally form part of an aerosol delivery system and in particular may form part of the reusable device and/or the consumable cartridge.

The claimed invention may advantageously provide adjustable operating characteristics to target certain characteristics which may be desirable to a user.

### BRIEF DESCRIPTION OF THE FIGURES

Embodiments of the disclosure will now be described, by way of example only, with reference to the accompanying drawings, in which:

FIGS. 1a-1c are schematic cross-section views of aerosol delivery systems in accordance with some embodiments of the disclosure;

FIGS. 2a-2b are schematic cross-section views of aerosol delivery subsystems in accordance with some embodiments of the disclosure; and

FIGS. 3a-c and 4a-c are schematic views of various aspects of different flow modifying members in accordance with some embodiments of the disclosure.

### DETAILED DESCRIPTION OF THE DISCLOSURE

Aspects and features of certain examples and embodiments are described herein. Some aspects and features of certain examples and embodiments may be implemented conventionally and these are not described in detail in the interest of brevity. It will thus be appreciated that aspects and features of apparatuses and methods discussed herein which are not described in detail may be implemented in accordance with any suitable conventional techniques.

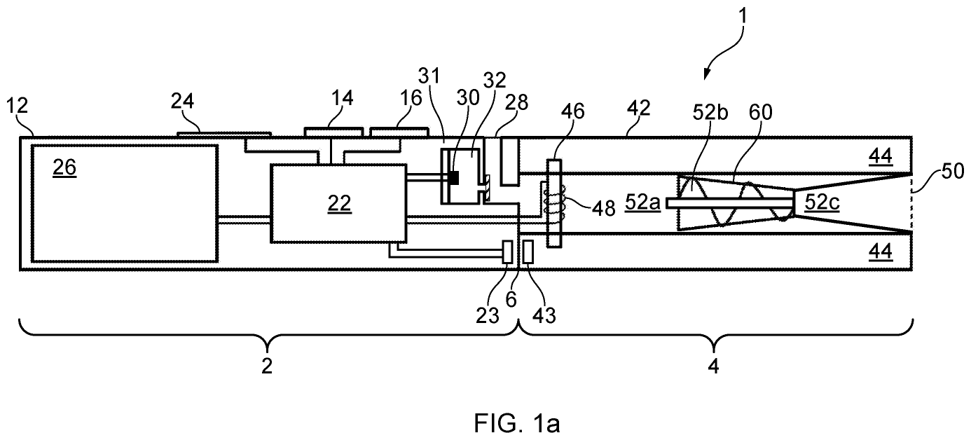

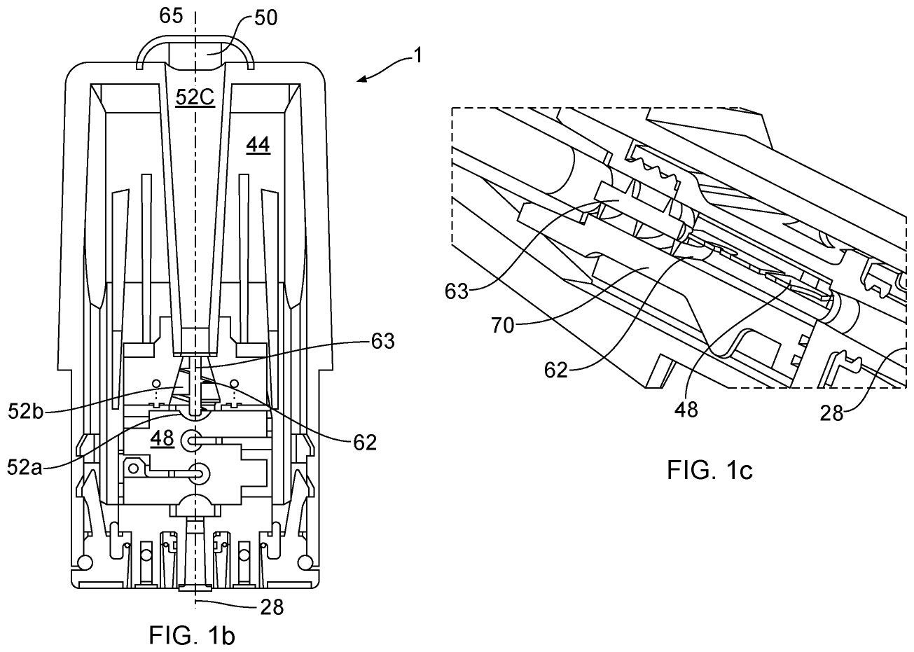

FIG. 1a is a cross-sectional view through an example aerosol delivery system 1 in accordance with certain embodiments of the disclosure. The aerosol delivery system 1 comprises two main components, namely a reusable part 2 and a replaceable/disposable consumable cartridge part 4. In normal use, the reusable part 2 and the cartridge part 4 are releasably coupled together at an interface 6. When the cartridge part 4 is exhausted or the user simply wishes to switch to a different cartridge part 4, the cartridge part 4 may be removed from the reusable part 2 and a replacement cartridge part 4 attached to the reusable part 2 in its place. The interface 6 provides a structural, electrical and airflow path connection between the two parts 2, 4 and may be established in accordance with conventional techniques, for example based around a screw thread, magnetic or bayonet fixing with appropriately arranged electrical contacts and openings for establishing the electrical connection and airflow path between the two parts 2, 4 as appropriate. The specific manner by which the cartridge part 4 mechanically mounts to the reusable part 2 is not significant to the principles described herein, but for the sake of a concrete example is assumed here to comprise a magnetic coupling (not represented in FIG. 1a). It will also be appreciated the interface 6 in some implementations may not support an electrical and/or airflow path connection between the respective parts 2, 4. For example, in some implementations an aerosol generator may be provided in the reusable part 2 rather than in the cartridge part 4, or the transfer of electrical power from the reusable part 2 to the cartridge part 4 may be wireless (e.g. based on electromagnetic induction), so that an electrical connection between the reusable part 2 and the cartridge part 4 is not needed. Furthermore, in some implementations the airflow through the electronic cigarette might not go through the reusable part 2, so that an airflow path connection between the reusable part 2 and the cartridge part 4 is not needed. In some instances, a portion of the airflow path may be defined at the interface between portions of the reusable part 2 and cartridge part 4 when these are coupled together for use.

The cartridge/consumable part 4 may in accordance with certain embodiments of the disclosure be broadly conventional. In FIG. 1a, the cartridge part 4 comprises a cartridge housing 42 formed of a plastics material. The cartridge housing 42 supports other components of the cartridge part 4 and provides the mechanical interface 6 with the reusable part 2. The cartridge housing 42 is generally circularly symmetric about a longitudinal axis along which the cartridge part 4 couples to the reusable part 2. In this example, the cartridge part 4 has a length of around 4 cm and a diameter of around 1.5 cm. However, it will be appreciated the specific geometry, and more generally the overall shapes and materials used, may be different in different implementations.

Within the cartridge housing 42 is a chamber or reservoir 44 that contains aerosol-generating material. In the example shown schematically in FIG. 1a, the reservoir 44 stores a supply of liquid aerosol generating material. In this example, the liquid reservoir 44 has an annular shape with an outer wall defined by the cartridge housing 42 and an inner wall that defines a fluid flow path or channel 52 through the cartridge part 4. The reservoir 44 is closed at each end with end walls to contain the aerosol generating material. The reservoir 44 may be formed in accordance with conventional techniques, for example it may comprise a plastics material and be integrally moulded with the cartridge housing 42.

The cartridge/consumable part 4 further comprises an aerosol generator 48 located towards an end of the reservoir 44 opposite to a mouthpiece outlet 50. It will be appreciated that in a two-part system such as shown in FIG. 1a, the aerosol generator 48 may be in either of the reusable part 2 or the cartridge part 4. For example, in some embodiments, the aerosol generator 48 (e.g. a heater, which may be in the form of a wick and coil arrangement as shown, a distiller, which may be formed from a sintered metal fibre material or other porous conducting material, or any suitable alternative aerosol generator) may be comprised in the reusable part 2, and is brought into proximity with a portion of aerosol generating material in the cartridge part 4 when the cartridge part 4 is engaged with the reusable part 2. In such embodiments, the cartridge part 4 may comprise a portion of aerosol generating material, and an aerosol generator 48 comprising a heater is at least partially inserted into or at least partially surrounds the portion of aerosol generating material as the cartridge part 4 is engaged with the reusable part 2.

In the example of FIG. 1a, a wick 46 in contact with the aerosol generator 48 extends transversely across the cartridge airflow path 52 with its ends extending into the reservoir 44 of the liquid aerosol generating material through openings in the inner wall of the reservoir 44. The openings in the inner wall of the reservoir 44 are sized to broadly match the dimensions of the wick 46 to provide a reasonable seal against leakage from the liquid reservoir 44 into the cartridge airflow path without unduly compressing the wick 46, which may be detrimental to its fluid transfer performance.

The wick 46 and aerosol generator 48 are arranged in the cartridge airflow path 52 such that a region of the cartridge airflow path 52 around the wick 46 and heater 48 in effect defines a vaporisation region for the cartridge part 4. Aerosol generating material in the reservoir 44 infiltrates the wick 46 through the ends of the wick extending into the reservoir 44 and is drawn along the wick by surface tension/capillary action (i.e. wicking). The aerosol generator 48 in this example comprises an electrically resistive wire coiled around the wick 46. In the example of FIG. 1a, the heater 48 comprises a nickel chrome alloy (Cr20Ni80) wire and the wick 46 comprises a glass fibre bundle, but it will be appreciated the specific aerosol generator configuration is not significant to the principles described herein. In use, electrical power may be supplied to the aerosol generator 48 to vaporise an amount of aerosol generating material (aerosol generating material) drawn to the vicinity of the aerosol generator 48 by the wick 46. Vaporised aerosol generating material may then become entrained in air drawn along the cartridge airflow path from the vaporisation region towards the mouthpiece outlet 50 for user inhalation.

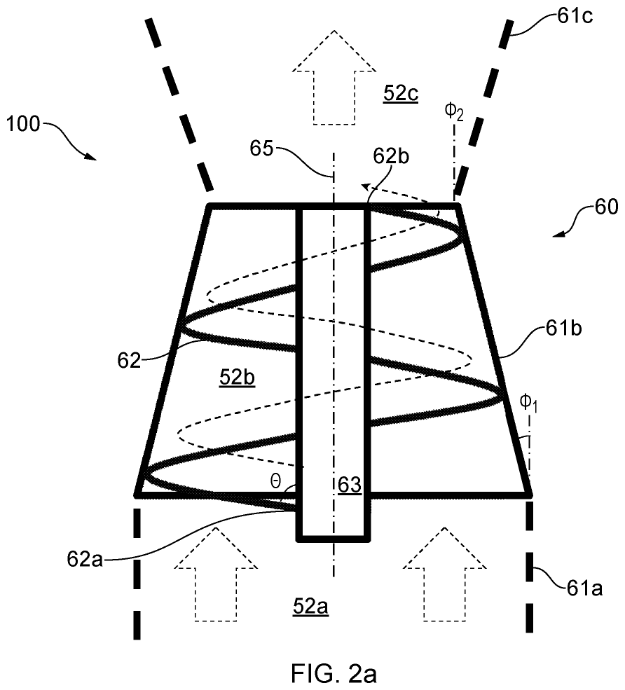

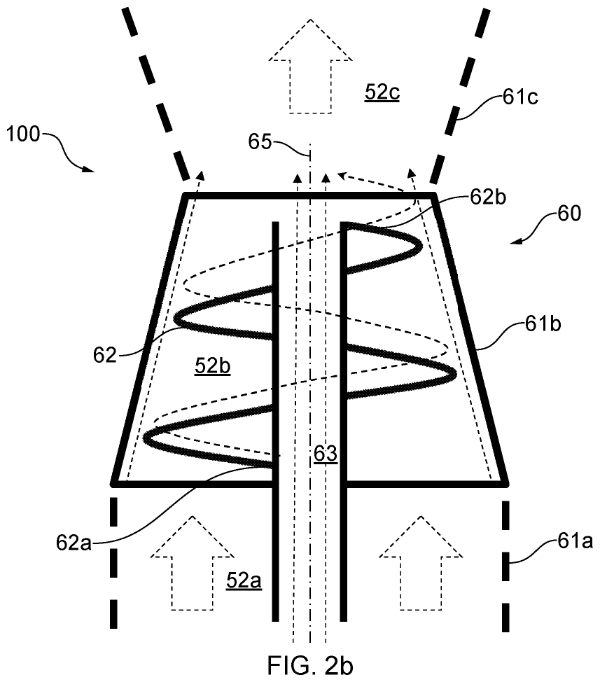

The aerosol delivery system 1 of FIG. 1a further comprises a flow modifying device 60 in the flow path or channel 52, which comprises a first, non-tapering (parallel walled) portion 52a downstream from the aerosol generator 48, a second, tapering (narrowing) portion 52b through the flow modifying device 60 and a third tapering (widening) portion 52c downstream from the flow modifying device 60. The flow modifying device 60 is configured to modify the flow of fluid (aerosol) from the vaporisation region along the flow path 52 towards the mouthpiece outlet 50, for user inhalation. The flow modifying device 60 is described in further detail with respect to FIGS. 2a-2b below.

The flow modifying device 60 may generally form part of an aerosol delivery system, such as two-part system or an all-in-one disposable system. Although the flow modifying device 60 is shown in FIG. 1a as sub-part of the consumable cartridge part 4 in the overall system 1, in other embodiments the reusable device part 2 may comprise the flow modifying device 60.

As noted above, the rate at which aerosol generating material is vaporised by the aerosol generator 48 will depend on the amount (level) of power supplied to the aerosol generator 48. Thus electrical power can be applied to the aerosol generator 48 to selectively generate aerosol from the aerosol generating material in the cartridge part 4, and furthermore, the rate of aerosol generation can be changed by changing the amount of power supplied to the aerosol generator 48, for example through pulse width and/or frequency modulation techniques.

The reusable part 2 comprises an outer housing 12 having with an opening that defines an air inlet 28 for the e-cigarette, a power source 26 (for example a battery) for providing operating power for the electronic cigarette, control circuitry/controller 22 for controlling and monitoring the operation of the electronic cigarette, a first user input button 14, a second user input button 16, and a visual display 24.

The outer housing 12 may be formed, for example, from a plastics or metallic material and in this example has a circular cross section generally conforming to the shape and size of the cartridge part 4 so as to provide a smooth transition between the two parts 2, 4 at the interface 6. In this example, the reusable part 2 has a length of around 8 cm so the overall length of the e-cigarette when the cartridge part 4 and the reusable part 2 are coupled together is around 12 cm. However, and as already noted, it will be appreciated that the overall shape and scale of an electronic cigarette implementing an embodiment of the disclosure is not significant to the principles described herein.

The air inlet 28 connects to an airflow path 51 through the reusable part 2. The reusable part airflow path 51 in turn connects to the cartridge airflow path 52 across the interface 6 when the reusable part 2 and cartridge part 4 are connected together. Thus, when a user inhales on the mouthpiece opening 50, air is drawn in through the air inlet 28, along the reusable part airflow path 51, across the interface 6, through the aerosol generation area in the vicinity of the aerosol generator 48 (where vaporised aerosol generating material becomes entrained in the air flow), along the cartridge airflow path 52, and out through the mouthpiece opening 50 for user inhalation.

The power source 26 in this example is rechargeable and may be of a conventional type, for example of the kind normally used in electronic cigarettes and other applications requiring provision of relatively high currents over relatively short periods. The power source 26 may be recharged through a charging connector in the reusable part housing 12, for example a USB connector.

First and/or second user input buttons 14, 16 may be provided, which in this example are conventional mechanical buttons, for example comprising a spring mounted component which may be pressed by a user to establish an electrical contact. In this regard, the input buttons may be considered input devices for detecting user input and the specific manner in which the buttons are implemented is not significant. The buttons may be assigned to functions such as switching the aerosol delivery system 1 on and off, and adjusting user settings such as a power to be supplied from the power source 26 to the aerosol generator 48. However, the inclusion of user input buttons is optional, and in some embodiments buttons may not be included.

A display 24 may be provided to give a user with a visual indication of various characteristics associated with the aerosol delivery system, for example current power setting information, remaining power source power, and so forth. The display may be implemented in various ways. In this example the display 24 comprises a conventional pixilated LCD screen that may be driven to display the desired information in accordance with conventional techniques. In other implementations, the display may comprise one or more discrete indicators, for example LEDs, that are arranged to display the desired information, for example through particular colours and/or flash sequences. More generally, the manner in which the display 24 is provided and information is displayed to a user using the display is not significant to the principles described herein. For example, some embodiments may not include a visual display and/or may include other means for providing a user with information relating to operating characteristics of the aerosol delivery system, for example using audio signalling, or may not include any means for providing a user with information relating to operating characteristics of the aerosol delivery system.

A controller 22 is suitably configured/programmed to control the operation of the aerosol delivery system 1 to provide functionality in accordance with embodiments of the disclosure as described further herein, as well as for providing conventional operating functions of the aerosol delivery system 1 in line with the established techniques for controlling such devices. The controller (processor circuitry) 22 may be considered to logically comprise various sub-units/circuitry elements associated with different aspects of the operation of the aerosol delivery system 1. In this example the controller 22 comprises power supply control circuitry for controlling the supply of power from the power source 26 to the aerosol generator 48 in response to user input, user programming circuitry 20 for establishing configuration settings (e.g. user-defined power settings) in response to user input, as well as other functional units/circuitry associated functionality in accordance with the principles described herein and conventional operating aspects of electronic cigarettes, such as display driving circuitry and user input detection circuitry. It will be appreciated that the functionality of the controller 22 can be provided in various different ways, for example using one or more suitably programmed programmable computer(s) and/or one or more suitably configured application-specific integrated circuit(s)/circuitry/chip(s)/chipset(s) configured to provide the desired functionality.

The functionality of the controller 22 is described further herein. For example, the controller 26 may comprise an application specific integrated circuit (ASIC) or microcontroller, for controlling the aerosol delivery device. The microcontroller or ASIC may include a CPU or micro-processor. The operations of a CPU and other electronic components are generally controlled at least in part by software programs running on the CPU (or other component). Such software programs may be stored in non-volatile memory, such as ROM, which can be integrated into the microcontroller itself, or provided as a separate component. The CPU may access the ROM to load and execute individual software programs as and when required.

The reusable part 2 comprises an airflow sensor 30 which is electrically connected to the controller 22. In most embodiments, the airflow sensor 30 comprises a so-called “puff sensor”, in that the airflow sensor 30 is used to detect when a user is puffing on the device. In some embodiments, the airflow sensor 30 comprises a switch in an electrical path providing electrical power from the power source 26 to the aerosol generator 48. In such embodiments, the airflow sensor 30 generally comprises a pressure sensor configured to close the switch when subjected to a particular range of pressures, enabling current to flow from the power source 26 to the aerosol generator 48 once the pressure in the vicinity of the airflow sensor 30 drops below a threshold value. The threshold value can be set to a value determined by experimentation to correspond to a characteristic value associated with the initiation of a user puff. In other embodiments, the airflow sensor 30 is connected to the controller 22, and the controller distributes electrical power from the power source 26 to the aerosol generator 48 in dependence of a signal received from the airflow sensor 30 by the controller 22. The specific manner in which the signal output from the airflow sensor 30 (which may comprise a measure of capacitance, resistance or other characteristic of the airflow sensor, made by the controller 22) is used by the controller 22 to control the supply of power from the power source 26 to the aerosol generator 48 can be carried out in accordance with any approach known to the skilled person.

In the example shown in FIG. 1a, the airflow sensor 30 is mounted to a printed circuit board 31 as described further herein, but this is not essential. The airflow sensor 30 may comprise any sensor which is configured to determine a characteristic of airflow in an airflow path 51 disposed between air inlet 28 and mouthpiece opening 50, for example a pressure sensor or transducer (for example a membrane or solid-state pressure sensor), a combined temperature and pressure sensor, or a microphone (for example an electret-type microphone), which is sensitive to changes in air pressure, including acoustical signals. The airflow sensor 30 is situated within a sensor cavity 32, which comprises the interior space defined by one or more chamber walls 34. The sensor cavity 32 may also be referred to herein as a sensor chamber 32 (these terms may be used interchangeably), and comprises a region internal to one or more chamber walls 34 in which an airflow sensor 30 can be fully or partially situated. In some embodiments, the airflow sensor 30 is mounted to a printed circuit board (PCB) 31, which comprises one of the chamber walls of a sensor housing comprising the sensor chamber/cavity 32.

A deformable membrane is disposed across an opening communicating between the sensor cavity 32 containing the sensor 30, and a portion of the airflow path disposed between air inlet 28 and mouthpiece opening 50. The deformable membrane covers the opening, and is attached to one or more of the chamber walls according to approaches described further herein.

As described further herein, the aerosol delivery system 1 comprises communication circuitry configured to enable a connection to be established with one or more further electronic devices (for example, a storage/charging case, and/or a refill/charging dock) to enable data transfer between the aerosol delivery system 1 and further electronic device(s). In some embodiments, the communication circuitry is integrated into controller 22, and in other embodiments it is implemented separately (comprising, for example, separate application-specific integrated circuit(s)/circuitry/chip(s)/chipset(s)). For example, the communication circuitry may comprise a separate module to the controller 22 which, while connected to controller 22, provides dedicated data transfer functionality for the aerosol delivery device. In some embodiments, the communication circuitry is configured to support communication between the aerosol delivery system 1 and one or more further electronic devices over a wireless interface. The communication circuitry may be configured to support wireless communications between the aerosol delivery system 1 and other electronic devices such as a case, a dock, a computing device such as a smartphone or PC, a base station supporting cellular communications, a relay node providing an onward connection to a base station, a wearable device, or any other portable or fixed device which supports wireless communications.

Wireless communications between the aerosol delivery system 1 and a further electronic device may be configured according to known data transfer protocols such as Bluetooth®, ZigBee, WiFi®, Wifi Direct, GSM, 2G, 3G, 4G, 5G, LTE, NFC, RFID. More generally, it will be appreciated that any wireless network protocol can in principle be used to support wireless communication between the aerosol delivery system 1 and further electronic devices. In some embodiments, the communication circuitry is configured to support communication between the aerosol delivery system 1 and one or more further electronic devices over a wired interface. This may be instead of or in addition to the configuration for wireless communications set out above. The communication circuitry may comprise any suitable interface for wired data connection, such as USB-C, micro-USB or Thunderbolt interfaces. More generally, it will be appreciated the communication circuitry may comprise any wired communication interface which enables the transfer of data, according to, for example, a packet data transfer protocol, and may comprise pin or contact pad arrangements configured to engage cooperating pins or contact pads on a dock, case, cable, or other external device which can be connected to the aerosol delivery system 1.

FIGS. 1b-1c will now be described highlighting the notable differences to FIG. 1a. In particular, the system of FIGS. 1b-1c comprises an aerosol generator 48 in the form of a distiller 48 (rather than a wick and coil in FIG. 1a) and the air inlet 28 is located at the base of the device, aligned along the same axis of extent 65 of the fluid channel 52, rather than on the side as in FIG. 1a. FIG. 1c also illustrates how the flow modifying device 60 is located within the system 1 by the flow modifying device 60 comprising an integral mounting bracket or aerosol generator (heater) cradle 70. The cradle 70 may have various functions, e.g. feeding liquid to the aerosol generator 48 to aerosolise it and/or providing an air flow into the system to “balance” the system (avoiding creating an internal vacuum). The cradle 70 comprises recesses and protrusions to locate the flow modifying device 60 axially and radially within the system with respect to the reservoir 44, the fluid channel 52 and the air inlet 28. As shown in FIG. 1b, the outlet 50 forms at least part of a mouthpiece through which a user can inhale aerosol in use.

FIGS. 2a-2b are schematic cross-section views of aerosol delivery subsystems 100 in accordance with some embodiments of the disclosure, showing more detail of the flow modifying device 60 in the flow channel 52 of the aerosol delivery systems 1 of FIGS. 1a-1c.

As shown in FIG. 2a, the flow modifying device 60 comprises a tapering fluid channel wall 61b defining the tapering fluid channel 52b through the device 60 (indicated by dashed arrows), wherein the fluid channel 52b tapers by narrowing in cross-section as shown along its length, in the direction of fluid flow through the device 60 (to the outlet 50) in use.

The flow modifying device 60 further comprises a flow modifying member 62 in the form of a wall 62 in the tapering portion 52b of the fluid channel 52, configured to impart a degree of rotation about the axis of extent 65 of the fluid channel to aerosol flowing in the fluid channel 52 in use. Thus, the flow modifying member 62 causes the flow of fluid (typically comprising air from the air inlet 28 with aerosol entrained therein from the aerosol generation region) in the tapering fluid channel 52b to be modified during use, in this case by introducing rotation, as indicated by the dashed arrow following the spiralling path of the flow modifying member 62. The flow modifying member 62 and/or the tapering fluid channel 52b may beneficially promote aerosol mixing, for example for mixing single or multiple fluids fed to the aerosol generator 48, and may cool the aerosol before it reaches the user. The flow modifying member 62 may also impact (increase/decrease) the particle size of the aerosol, altering the ‘feel’for the user.

The combination of the tapering fluid channel 52b with a flow modifying member 62 configured to impart a degree of rotation about the axis of extent 65 of the fluid channel 52b to aerosol flowing in the fluid channel in use provides an advantageously compact fluid channel 52 which minimises condensate forming on the fluid channel walls 61. Accordingly, this allows for a reduction in size of the overall system, reducing material use and costs, without impacting performance or the user experience. Moreover, introducing rotation (swirl) into the aerosol fluid flow can be desirable for the user.

In the FIG. 2a embodiment, the flow modifying member 62 is a single, continuous spiralling member 62 formed separately from the fluid channel wall 61. The flow modifying member 62 extends from a spine or shaft 63 which extends along the central axis of extent 65 of the fluid channel 52, and thus the flow modifying member 62 may comprise an insert for the fluid channel 52b, which may be user-replaceable. The shaft 53 may be a solid shaft or a tubular shaft allowing fluid flow therethrough, which can provide a mixture of fluid flow with and without imparted rotation, discussed further below with respect to FIG. 2b.

In FIG. 2a, the fluid channel 52b tapers by the wall 61b narrowing in cross-section along its length as shown and in some embodiments tapers by the channel 52b reducing in cross-sectional area along its length. In FIG. 2a, the flow modifying member 62 follows the same taper and each spiral substantially contacts the wall 61b, thus minimising air flow along the tapering sides of the channel 52b, between the wall 61b and the flow modifying member 62. In some embodiments, the wall 61b and/or the flow modifying member 62 may comprise a seal to reduce such bypass air flow further.

In FIGS. 2a-2b, the tapering fluid channel 52 b has a major diameter of around 6 mm, a minor diameter of around 3 mm and a length of around 15 mm. The wall 62 has a thickness of around 0.5 mm. The flow modifying member 62 may comprise an auger and/or may span the full width (diameter) of the channel 52, as shown in FIG. 2a, or alternatively may only span a partial width (diameter) of the channel 52, e.g. up to any of 50%, 60%, 70%, 80%, 90% or 95% of the total width, allowing fluid to flow around (bypass) the flow modifying member 62, discussed further with respect to FIG. 2b below.

In the FIG. 2a embodiment, a first (upstream) end 62a of the flow modifying member 62 extends into the aerosol generation chamber/area, containing the aerosol generator 48, said area having parallel fluid channel walls 61a defining the fluid channel 52a in the aerosol-generating area. The extension of the flow modifying member 62 into the aerosol generation chamber can remove latent heat asap from the heated aerosol, to reduce condensate forming. The sooner the angle of attack impinges on the aerosol generation area, the quicker the latent heat can be removed from the recently-heated aerosol. The flow modifying member 62 aims to ‘swirl’ the aerosol when it is at its hottest, to reduce the condensate forming. The extension into the aerosol generation chamber therefore forms a collection point for aerosol and not condensate—picking up that aerosol as quickly as possible. This can also limit (reduce) the length of the mouthpiece required.

A second (downstream) end 62b of the flow modifying member 62 terminates where the fluid channel wall 61 transitions from a narrowing wall 61b to a diverging (widening) wall 61c which defines a downstream widening fluid channel 52c, towards the outlet 50. Accordingly, in FIG. 2a, the second, narrowing portion 52b of the fluid channel 52 surrounds the flow modifying member 62 whilst the third, widening portion 52c of the fluid channel 52 does not surround the flow modifying member 62.

In some embodiments, the flow modifying member 62 may be secured to or integrally formed with the fluid channel wall 61, or may extend from an inner surface of the fluid channel wall 61. The flow modifying member 62 may be of any shape suitable for imparting the desired degree of rotation, such as curved or helical, e.g. with a constant or variable pitch, and may comprise multiple distinct flow modifying members.

In some embodiments, the second, narrowing portion 52b of the fluid channel 52 comprises the fluid channel wall 61b inclined at a non-zero angle φ₁ to the axis of extent of the fluid channel 52, wherein the non-zero angle φ₁ is within the range of 5 to 45°, 10 to 40°, 15 to 35° or 20 to 30°; and/or the third, widening portion 52c of the fluid channel comprises the fluid channel wall 61c inclined at a non-zero angle φ₂ to the axis of extent of the fluid channel 52, wherein the non-zero angle φ₂ is within the range of 5 to 45°, 10 to 40°, 15 to 35° or 20 to 30°. The inclination angles φ₁ and φ₂ may be the same or differ. In the embodiments of FIGS. 2a and 2b, φ₁ is approximately 20°, whilst φ₂ is approximately 30°.

FIG. 2b will now be described highlighting key differences to FIG. 2a. In FIG. 2b, the flow modifying member 62 again has the same taper gradient as the fluid channel 52b (=φ₁), but each spiral does not contact the wall 61b, thus permitting bypass air flow along the tapering sides of the channel 62b (see dashed arrows). Furthermore, in FIG. 2b, the shaft 63 is tubular rather than solid, permitting air flow therethrough (see dashed arrows), and extends into the upstream fluid channel 52a to locate the flow modifying member 62 in the fluid channel, e.g. by securing to or extending from the aerosol generator 48, or securing to or extending from the upstream wall 61a, such as securing by interference fit with a horizontal connecting element between the shaft 63 and the wall 61a. Finally, the wall 62 of the flow modifying member 62 does not extend into the aerosol generation area in the straight, parallel sided portion of the fluid channel 52a—instead, the upstream end of the wall 62a is downstream from the straight, parallel sided portion of the fluid channel 52a whilst the downstream end 62b terminates upstream from the diverging portion of the fluid channel 52c.

In further embodiments (not shown), the flow modifying member 62 has a different taper gradient to the fluid channel 52b. The tapering of the flow modifying member 62 may be greater than the fluid channel 52b, permitting air flow along the tapering sides of the channel 62b, and/or may be variable, including any combination of zero taper (straight-through), diverging and converging, as may be desired to impact fluid flow to the user.

In the FIG. 2a-2b embodiments, the flow modifying member 62 provides two complete spirals between its ends 62a, 62b and has a surface inclined at a non-zero angle Θ of approximately 75° to the axis of extent 65 of the fluid channel 52. This non-zero angle Θ may be considered the angle of attack to incoming aerosol. In FIG. 1a, Θ is shown as around 45°. Generally, Θ may be within the ranges of 10 to 80°, 20 to 70°, 30 to 60° or 40 to 50°, preferably 55-75, 60-70°. In one particular embodiment (not shown), the flow modifying member 60 comprises two or more co-spiralling flow modifying members 60 which spiral in phase, out of phase or with a phase difference.

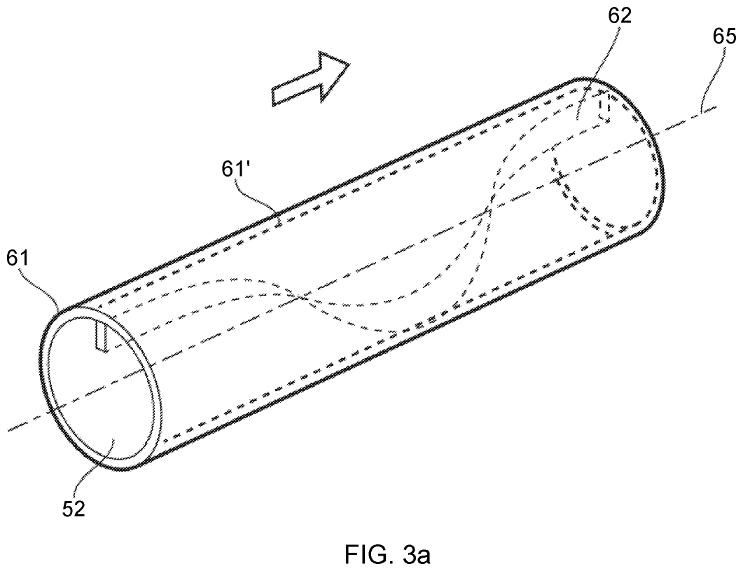

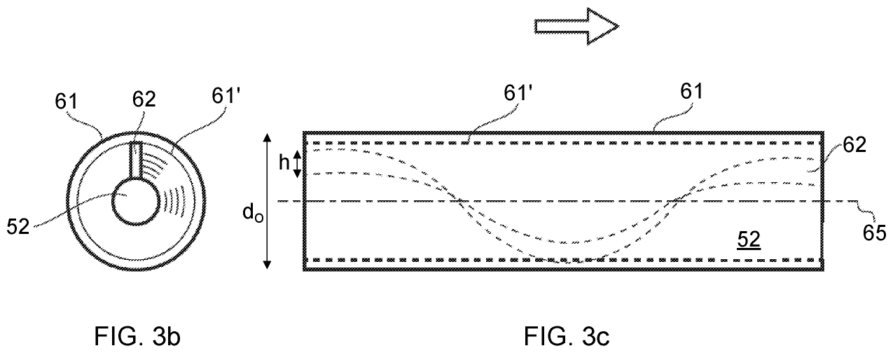

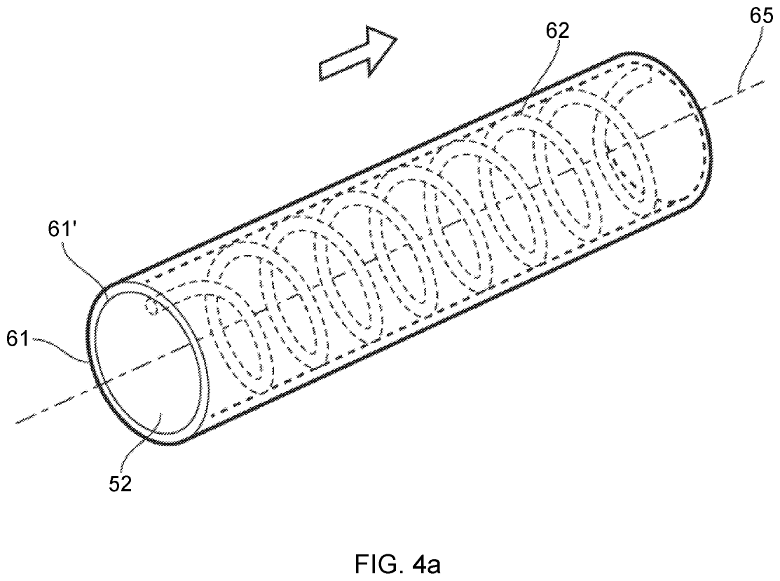

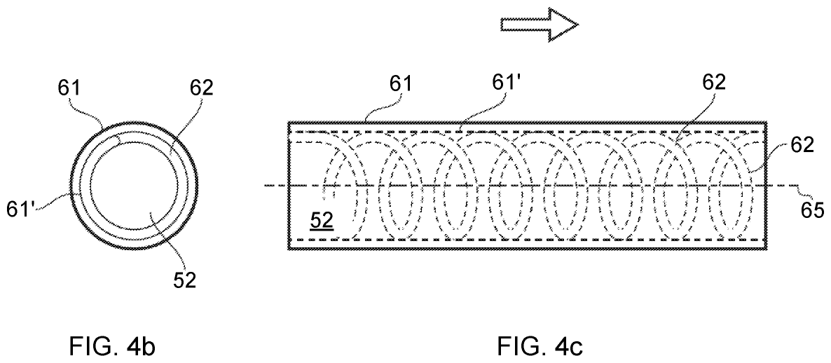

FIGS. 3a-c and 4a-c are schematic views of various aspects of different flow modifying members 60 as alternative methods for introducing rotation. In these figures, the fluid channel 52 is shown as a straight (not tapering) fluid channel 52 for ease of representation.

FIG. 3a schematically represents a perspective view of the fluid channel wall 61 with elements hidden behind the outer surface of the fluid channel wall 61 shown in dashed line. FIG. 3b schematically represents an end view of the fluid channel wall 61, in this example the left-hand end of the representation of FIG. 3a. FIG. 3c schematically represents a side view of the fluid channel wall 61. The direction of normal fluid flow in use is indicated by an arrow. As noted above, for ease of representation, the fluid channel wall 61 is shown as comprising a generally cylindrical (not tapering) shape with structural features associated with the coupling of the fluid channel 52 to the aerosol generation area in the first portion of the fluid channel 52a and the outlet 50 not shown for simplicity.

Also represented in FIGS. 3a to 3c is an inner wall 61′ of the fluid channel wall 61, which defines an outer surface of the fluid channel 52 through which fluid flows when the aerosol delivery system 1 is in use. As schematically represented in these figures, the fluid channel 52 includes the flow modifying member 62 extending into the fluid channel 52 from a part of the inner wall 61′. In this example, the flow modifying member 62 is in the form of a wall 62 running the length of the portion of the fluid channel 52 represented in FIG. 3a along a generally spiralling/helical path, completing around one turn.

The helical/spiralling path of the flow modifying member 62 extends into the fluid channel 52 with a surface facing fluid drawn along the channel 52 and inclined at a non-zero angle to the axis of extent 65 of the channel 52 (i.e. an axis corresponding generally to the direction of fluid flow in use). As in FIG. 1a, this causes fluid passing along the channel 52 to be deflected about the central axis of the channel (in this example in a clockwise direction as viewed from the upstream end), thereby imparting a degree of rotation about the axis of extent 65 of the channel 52 to fluid flowing through the channel 52.

The degree of rotation will depend on various factors, such as the size of the flow modifying member 62 (i.e. how far it extends into the channel 52 radially—height h shown in FIG. 3c, its inclination angle to the axis of extent 65 and the number of flow modifying members). In the example represented in FIGS. 3 a to 3 c, the channel 52 has an outer diameter do of around 5 mm, a wall thickness of 0.25 mm and the protrusion has a height h of around 1 mm, i.e. less than quarter the total diameter. In this example, the flow modifying member 62 presents a relatively shallow angle to incoming air, for example around 15°. Furthermore, in this example there is only one flow modifying member 62.

If a greater degree of airflow modification (i.e. more rotation) is desired, a greater number of flow modifying members could be added with an appropriate azimuthal offset from the flow modifying member wall 62 represented in FIGS. 3a to 3 c (e.g. 180 degrees offset for one further wall, 120 degrees offset for each of two further walls, etc.). Also, the extent (height h) of the wall(s) or other flow modifying members 62 into the channel 52 could be increased to increase the modification to the fluid flow. Furthermore, a tighter spiral (i.e. more turns along the length of the channel 52) could be used to provide an increase in the deflection angle presented to fluid flowing in the channel 52. For example, the deflection angle may be selected from the group comprising: at least 10 degrees; at least 20 degrees; at least 30 degrees; at least 40 degrees; at least 50 degrees; at least 60 degrees; at least 70 degrees; and at least 80 degrees.

To introduce a smaller degree of rotation, the flow modifying member wall 62 could be made smaller, or it may be broken into a number of non-continuous portions along the helical path. More generally, it will be appreciated there are many parameters for the configuration of the one or more flow modifying members 62 which could be adjusted to provide a desired degree of rotation. An appropriate degree of rotation for any given implementation could be determined empirically, for example, by testing the performance of different configurations. In some respects, the approaches of introduction of rotation into fluid flow along the channel 52 may be considered to providing a rifling effect.

FIGS. 4a-4c are generally similar to, and will be understood from, FIGS. 3a-3c. However, whereas in the example of FIGS. 3a-3c, the fluid flow modification (rotation) is achieved using a flow modifying member 62 comprising a helical wall 62 integrally formed with the fluid channel wall 61, in the example of FIGS. 4a-4c, the flow modifying member 62 comprises a helical spring-shaped structure 62 which is inserted into the fluid channel 52 which is defined by an otherwise smooth inner wall 61′. In this example, the helical spring-shaped member 62 comprises a conventional spring having an appropriate outer diameter and thickness (gauge). In this regard, the thickness of the spring flow modifying member 62 providing the protrusion in FIGS. 4a-4c is, in this example, less than the height h of the wall flow modifying member 62 in FIGS. 3a-3c, but the spring flow modifying member 62 is arranged to present a steeper angle to incoming fluid (i.e. arranged on a tighter spiral with more turns) and so may introduce a broadly corresponding degree of rotation to fluid flowing in the channel 52.

In a further variant (not shown separately, but that can be understood from FIGS. 4a-c), the flow modifying member 62 comprises a spiralling spring-shaped tube 62, where the tube 62 receives the fluid flow and imparts a degree of rotation to fluid flowing therein, rather than the flow modifying member 62 being a protrusion for deflecting air flow as outlined above. In this embodiment, the tubular flow modifying member 62 may have a greater inner diameter with less tight spiralling than the thickness of the spring variant depicted in FIGS. 4a-4c, to provide a less tortuous path for fluid flow therein. In any event, and as discussed above, an appropriate configuration providing a desired degree of fluid flow modification can be established through empirical testing, for example by assessing the performance using spring or tubular flow modifying members 62 of different dimensions.

In some embodiments, the flow modifying member 62 provides two complete spirals between its ends 62a, 62b. In other embodiments, any number and any combination of complete and incomplete (partial) spirals may be provided.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention.

Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

Protection may be sought for any features disclosed in any one or more published documents referenced herein in combination with the present disclosure.

1. An aerosol delivery subsystem for an aerosol delivery system, comprising:
a fluid channel wall defining a fluid channel, wherein at least a portion of the fluid channel tapers along its length; and
a flow modifying member in the tapering portion of the fluid channel, configured to impart a degree of rotation about an axis of extent of the fluid channel to aerosol flowing in the fluid channel in use.

2. The subsystem of claim 1, wherein the flow modifying member is a curved, spiralling or helical member configured to impart the degree of rotation about the axis of extent of the fluid channel to aerosol flowing in the fluid channel in use.

3. The subsystem of claim 1, wherein:
the flow modifying member and the fluid channel wall are integrally formed; or
the flow modifying member is formed separately from the fluid channel wall and comprises an insert for the fluid channel.

4. The subsystem of claim 1, wherein the flow modifying member comprises a wall, a spring, a tube and/or an auger.

5. The subsystem of claim 1, wherein the flow modifying member:
a. extends from an inner surface of the fluid channel wall; and/or
b. extends from a shaft extending along or parallel to the central axis of extent of the fluid channel.

6. The subsystem of claim 1, wherein the fluid channel wall comprises the flow modifying member.

7. The subsystem of claim 1, wherein the flow modifying member extends into an aerosol generation area.

8. The subsystem of claim 1, wherein the flow modifying member:
a. provides at least two complete spirals; and/or
b. has a surface inclined at a non-zero angle to the axis of extent of the fluid channel, wherein the non-zero angle is within the range of 10 to 80°, 20 to 70°, 30 to 60° or 40 to 50°.

9. The subsystem of claim 1, wherein the flow modifying member comprises a single continuous flow modifying member or multiple distinct flow modifying members.

10. The subsystem of claim 1, wherein the flow modifying member comprises two or more co-spiralling flow modifying members which spiral in phase, out of phase or with a phase difference.

11. The subsystem of claim 1, wherein a cross-sectional area of the fluid channel tapers along its length toward the outlet.

12. The subsystem of claim 1, wherein the fluid channel comprises:
a. a narrowing portion which narrows along its length; and
b. a widening portion which widens along its length.

13. The subsystem of claim 1, wherein:
a. the narrowing portion of the fluid channel surrounds the flow modifying member; and/or
b. the widening portion of the fluid channel does not surround the flow modifying member.

14. The subsystem of claim 12, wherein:
a. the narrowing portion of the fluid channel comprises the fluid channel wall inclined at a non-zero angle to the axis of extent of the fluid channel, wherein the non-zero angle is within the range of 5 to 45°, 10 to 40°, 15 to 35° or 20 to 30°; and/or
b. the widening portion of the fluid channel comprises the fluid channel wall inclined at a non-zero angle to the axis of extent of the fluid channel, wherein the non-zero angle is within the range of 5 to 45°, 10 to 40°, 15 to 35° or 20 to 30°.

15. The subsystem of claim 1, wherein:
a. the subsystem further comprises a housing for receiving an aerosol generator and/or an aerosol-generating material; and/or
b. the fluid channel comprises an inlet and an outlet, wherein the outlet forms at least part of a mouthpiece through which a user can inhale aerosol in use.

16. The subsystem of claim 1, further comprising:
a. an aerosol generator; and/or
b. a cartridge or cartomizer housing an aerosol-generating material for generating aerosol for inhalation by a user; and/or
c. a housing comprising the inlet and/or outlet; and/or
d. a controller; and/or
e. a power source.

17. An aerosol delivery system comprising the subsystem of claim 1 and:
a. an aerosol generator; and/or
b. a cartridge or cartomizer housing an aerosol-generating material for generating aerosol for inhalation by a user; and/or
c. a housing comprising the inlet and/or outlet; and/or
d. a controller; and/or
e. a power source.

18. A flow modifying device for an aerosol delivery system, the device comprising:
a fluid channel wall defining a fluid channel, wherein at least a portion of the fluid channel tapers along its length; and
a flow modifying member in the tapering portion of the fluid channel, configured to impart a degree of rotation about an axis of extent of the fluid channel to aerosol flowing in the fluid channel in use.

19. (canceled)

20. (canceled)

21. A method of modifying fluid flow along a flow channel defined by a fluid channel wall, wherein at least a portion of the fluid channel tapers along its length, the method comprising:
imparting a degree of rotation about the axis of extent of the fluid channel to aerosol flowing in the tapering portion of the fluid channel.
